(19) United States
(12) Patent Application Publication
(10) Pub. No.: US 2016/0250295 A1
(43) Pub. Date: Sep. 1, 2016

(54) FORMULATION FOR GONADOTROPINS

(71) Applicant: CADILA HEALTHCARE LIMITED, Ahmedabad, Gujarat (IN)

(72) Inventors: Chintan PATEL, Ahmedabad, Gujarat (IN); Mukesh MAHAJAN, Ahmedabad, Gujarat (IN); Sanjay BANDYOPADHYAY, Ahmedabad, Gujarat (IN); Sanjee Kumar MENDIRATTA, Ahmedabad, Gujarat (IN); Bhatt CHANDRESH, Ahmedabad, Gujarat (IN)

(73) Assignee: CADILA HEALTHCARE LIMITED, Ahmedabad, Gujarat (IN)

(21) Appl. No.: 15/030,527

(22) PCT Filed: Oct. 31, 2014
(86) PCT No.: PCT/IN2014/000691

§ 371 Date: Apr. 19, 2016

(30) Foreign Application Priority Data
Nov. 12, 2013 (IN) 3559/MUM/2013

(51) Int. Cl. A61K 38/24; A61K 47/12; A61K 47/18; A61K 47/02; A61K 47/10; A61K 9/08

(52) U.S. Cl. CPC A61K 38/24; A61K 47/10; A61K 9/08; A61K 47/183; A61K 47/02; A61K 47/12

(57) ABSTRACT

The present invention relates to a stable composition for gonadotropins. It provides a composition useful for stabilization of gonadotropins while preventing aggregation, dissociation, fragmentation and formation of oxidized species variants in solution for injection. Thus, it prevents instability of protein or polypeptide molecules caused due to aggregation or fragmentation or oxidation during or after formulation. Also, it provides a pharmaceutical composition of gonadotropins, which can be therapeutically used for the treatment of various indications either in single-dose form or in multi-dose form.

### FIELD OF THE INVENTION

The present invention relates to stable pharmaceutical compositions of gonadotropins. It provides a formulation composition useful for stabilization of gonadotropins by preventing aggregation, dissociation, fragmentation and formation of oxidized species variants during and after formulation. It also provides a pharmaceutical composition of gonadotropins, which can be therapeutically used for the treatment of various indications either in single-dose form or in multi-dose form.

### BACKGROUND OF THE INVENTION

Therapeutic proteins or polypeptides pose a number of challenges for pharmaceutical scientists regarding their formulation and delivery. Maintaining the physical and chemical stability of protein or polypeptide molecules in solution is important to retain the biologically active conformation of the molecule, which results in providing the desired level of potency and safety of the pharmaceutical preparation for injection comprising the protein or polypeptide molecules. Lack of physical and chemical stability may lead to significant degradation or irreversible modifications of protein or polypeptide molecules during processing, manufacturing, transportation and storage. Protein aggregation or fragmentation in pharmaceutical preparation is associated with loss of efficacy, altered pharmacokinetics, reduced stability, limited product shelf-life, and induction of unwanted immunogenicity. Aggregation or dissociation or fragmentation or oxidation of protein or polypeptide molecules in pharmaceutical preparation severely affects the potency of the drug product. Pharmaceutical preparation comprising such functionally compromised molecule significantly alters the efficacy, bioavailability, tissue distribution pattern and pharmacokinetic profile of the drug product with higher risk of immunogenicity. In pharmaceutical preparation of protein drag product, a number of excipients have been used with varying success to reduce such protein degradation or modification. However, each excipient has its own limitations, and in some cases, the more effective ones are less amenable to inclusion in final formulation. Therefore, it is always challenging to establish stable formulation of sensitive protein or polypeptide molecules with a mixture of suitable inactive ingredients or excipients of interest, for pharmaceutical use.

Here, the present invention provides pharmaceutical composition of proteins or polypeptides, preferably gonadotropins, which provides stable formulation of the said molecules for therapeutic use either in single-dose or multi-dose form. Follicles stimulating hormone (FSH), Luteinizing hormone (LH), Human chorionic gonadotropin (hCG) etc. are glycoproteins in nature and composed of two subunits, alpha and beta, which remain held together by non-covalent forces in protein structure. Glycosylation occurs on both alpha and beta subunits at specific sites on the polypeptide backbone. The alpha subunit is identical among the specified gonadotropins, while beta subunit is different for each of these glycoproteins. The beta unit is responsible for the specificity of the biological activity. The subunit alone has no known biological activity. It is the formation of heterodimer that provides the biological activity of the protein molecules. The present invention aims to deliver novel composition for therapeutically effective amount of FSH or its variants, which provides stable formulation of FSH or its variants for pharmaceutical use either in single-dose, or multi-dose form. Follitropin alpha (recombinant human follicle-stimulating hormone; follitropin alfa) is a recombinant form of follicle-stimulating hormone (FSH), an endogenous gonadotrophin. Follitropin beta is a human follicle stimulating hormone (FSH) preparation of recombinant DNA origin, which consists of two non-covalently linked, non-identical glycoproteins designated as the alpha- and beta- subunits. The alpha- and beta- subunits have 92 and 111 amino acids. The alpha subunit is glycosylated at Asn 51 and Asn 78 while the beta subunit is glycosylated at Asn 7 and Asn 24.

EP 1928413 application provides an aqueous formulation of a human follicle stimulating hormone (hFSH), comprising a therapeutically effective amount of hFSH, and glycine, methionine, a non-ionic surfactant and a phosphate buffer as stabilizers. Non-ionic surfactants are selected from poloxamer and polysorbates, preferably polysorbates.

WO2011/108010 provides a formulation comprising human gonadotropin or its variant with buffer system selected from the group consisting of acetate, lactate, carbonate and bicarbonate or their combination at, a pH in the range of 6.5 to 9.0. Further, it includes ampholytes, sugars, polysorbates, antioxidants and preservatives.

U.S. Pat. No. 5,929,028 discloses a liquid gonadotropin-containing formulation characterized in that the formulation comprises a gonadotropin and stabilizing amounts of a polycarboxylic acid or a salt, thereof, and of a thioether compound.

We, hereinafter, provide various formulations of the desired proteins or polypeptides, preferably gonadotropins, more preferably FSH or its variants, in which the said proteins or polypeptides remain adequately stable without undergoing further aggregation or dissociation or fragmentation or oxidation or any other modifications during and after formulation. The formulations disclosed, hereinafter, can be stored for longer period of time, under suitable storage conditions and provide better stability.

### SUMMARY OF THE INVENTION

The present invention provides a liquid , stable formulation containing therapeutic amount of gonadotropins, preferably FSH or its variants for the purpose of single-use or multiple-use.

In one aspect, the present invention, provides a stable liquid formulation containing a therapeutic amount of FSH or its suitable variants and suitable excipients, selected from suitable buffers, stabilizer(s), antioxidants, preservatives and other excipients optionally selected from suitable surfactants, amino acids, and tonicity agents

In another aspect, the present invention provides a process for preparing a stable liquid formulation of FSH or its suitable variants with suitable buffer(s), stabilizer(s), antioxidants, preservatives and other excipients optionally selected from suitable surfactants, amino acids, and tonicity agents.

In further aspect, such formulations can also be, optionally lyophilized. Lyophilization can be performed by a skilled person using the techniques available in the art, which includes various steps like freezing, annealing, primary drying and secondary drying.

In yet another aspect, the present invention provides a liquid stable formulation, which comprises of about 5 μg/mL to 200 μg/mL of FSH or its variants and suitable buffers at a concentration of about 5 mM to 100 mM, suitable stabilizers in a concentration of about 0.005% to 10%, optionally suitable surfactants at a concentration of about 0.001% to 5%, antioxidants at a concentration of about 0.001% to 1% and optionally, preservatives at a concentration of about 0.01% to 1%, for therapeutic use either in single-dose or multi-dose form.

In another aspect, the present invention provides a liquid stable formulation, which optionally can be in lyophilized form comprising of about 5 μg/mL to 200 μg/mL of FSH or its variants and suitable buffers at a concentration of about 5 mM to 100 mM, optionally suitable stabilizers with a concentration of about 0.005% to 10%, optionally suitable surfactants at a concentration of about 0.001% to 5%. The said lyophilized preparation is reconstituted in suitable diluent, preferably, in the presence of suitable preservatives at a concentration of about 0.01% to 1%, for therapeutic use either in single-dose or multi-dose form.

In one of the embodiments, the present invention provides a liquid formulation buffered between pH 5 to 9.

In another embodiment, the present invention provides a liquid formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In another embodiment, the present invention provides a liquid formulation which stabilizes the protein or polypeptide molecule in solution by preventing any further degradation of the desired protein or polypeptide, during and after formulation. Generally, a stable formulation is the one which retains the physical stability and chemical stability and/or biological activity over a period of time, upon storage.

In a further embodiment, the present invention provides a liquid stable formulation of FSH or its variants, which can be therapeutically used for the relevant indications.

### DETAILED DESCRIPTION OF THE PRESENT INVENTION

The present invention provides novel liquid stable formulation, which can optionally be lyophilized, comprising, of suitable amount of therapeutic protein(s), preferably gonadotropins in suitable buffer(s), one or more suitable stabilizers, and other excipients, which are selected from antioxidants, preservatives and other excipients optionally selected from suitable surfactants, amino acids, and tonicity agents. The present formulation stabilizes the gonadotropins during and after formulation and prevents any further degradation or modification of protein or polypeptide, while maintaining the active biological conformation of the protein or polypeptide during and after formulation. In such embodiment, the protein is gonadotropin. In such embodiment the gonadotropin is derived from either urine or can be produced by recombinant technology. In a preferred embodiment the gonadotropin is selected from FSH, LH, hCG and combination thereof. In a more preferred embodiment, gonadotropin is FSH or its variants.

In some embodiments, the FSH or its variant is generally present in a therapeutic amount of up to 200 μg/mL. In a preferred embodiment the therapeutic amount is about 5 μg/mL to 100 μg/ml. In a more preferred embodiment the therapeutic amount is about 5 μg/mL to 50 μg/mL.

The liquid formulation comprises a suitable buffer along with other pharmaceutically acceptable excipients, which stabilizes the pharmaceutical preparation. Suitable buffers, which can be used are selected from those, which are known in the art and can be found in literature. In an embodiment, the suitable buffers comprise but are not limited to histidine, arginine, citrate, succinate, acetate, phosphate, tromethamine buffers and the like or their suitable mixtures such as citrate-phosphate and the like.

In a preferred embodiment, the suitable buffer comprises of a phosphate buffer or a succinate buffer.

The buffers are generally used in concentrations of about 5 mM to 100 mM. In a preferred embodiment, the buffer concentration is about 10 mM to 50 mM.

In an embodiment, the liquid formulation maintains a pH value ranging from pH 5 to about pH 9 depending on the FSH or its variant being used. In a preferred embodiment, the buffer used maintains the pH of the formulation in the range of about pH 6 to pH 8. In a more preferred embodiment, the pH is maintained to about pH 7.

The liquid formulation further comprises suitable surfactants, which are pharmaceutically acceptable excipients used to protect the protein formulations against various stress conditions, like agitation, shearing, exposure to high temperature etc., and reduce the surface interaction e.g., liquid-air or liquid-solid interfaces, during and after formulation. The suitable surfactants include but are not limited to polyoxyethylensorbitan fatty acid esters (Tween), polyoxyethylene alkyl ethers (e.g. Brij), alkylphenylpolyoxyethylene ethers (e.g. Triton-X), polyoxyethylene-polyoxypropylene copolymer (e.g. Poloxamer, Pluronic), octanoic acid (caprylate), sodium dodecyl sulphate (SDS) and the like. In a preferred embodiment, the suitable surfactant is polyoxyethylenesorbitan-fatty acid esters (Tweens). In a more preferred embodiment, the polyoxyethylenesorbitan-fatty acid esters are polysorbate 20, (sold under the trademark Tween 20™) and polysorbate 80 (sold under the trademark Tween 80™). In another preferred embodiment, the suitable surfactant is polyethylene-polypropylene copolymers, which are sold under the names Pluronic (R) F68 or Poloxamer 188™. In another preferred embodiment, the suitable surfactant is alkylphenolpolyoxyethylene esters, which are sold under the trade name Triton-X.

The surfactants are generally used in concentrations of about 0.001% to 5%. In a preferred embodiment, surfactant concentration is about 0.01% to 1%.

The liquid formulation further comprises one or more pharmaceutically acceptable or suitable stabilizer(s), which protect the active pharmaceutical ingredient from, chemical and/or physical degradation during processing, manufacturing, transportation, storage and application. In an embodiment, the stabilizers include but are not limited to suitable sugars, amino acids, polyols, polyethylene glycols (PEGs), polyethyleneimine, cyclodextrines and the like or suitable derivative or mixtures, thereof.

In one such embodiment, the sugar is a monosaccharide or an oligosaccharide. Monosaccharide sugars include but are not limited to glucose, fructose, galactose, mannose, sorbose, ribose, deoxyribose, dextran, dextrin and the like or amino sugars, like neuraminic acid or N-acetyl glucosamine and the like. An oligosaccharide includes but is not limited to sucrose, trehalose, lactose, maltose and raffinose and the like or suitable mixtures, thereof.

In another embodiment the polyols which can be used as stabilizers include but are not limited to mannitol, sorbitol, glycerol, arabitol, polyethylene glycol, propylene glycol and the like or suitable combinations thereof. In a preferred embodiment the suitable polyol is sorbitol or mannitol.

In another embodiment, polyethyleneimine can also be used as a stabilizer. In another preferred embodiment, the stabilizer is selected from sugar, polyol and suitable combination thereof. In an embodiment the stabilizer is present in amount about 0.005% to about 10%.

In a more preferred embodiment, the stabilizer is Polyethylene glycol (PEG) or Polyethyleneimine. In the present invention, polyethylene glycol having molecular weight in the range of 200 Dalton to 40,000 Dalton can be used. In a preferred embodiment, the formulation according to the present invention contains polyethylene glycol having molecular weight in the range of 200 Dalton to 10,000 Dalton. In a preferred embodiment, polyethylene glycol is present in amount about 0.005% to about 10%.

In another embodiment, cyclodextrines or derivatives thereof, which can be used as stabilizers, include but are not limited to α-cyclodextrin, β-cyclodextrin, γ-cyclodextrin, or their hydroxypropylated, hydroxyethylated, ethylated or methylated derivatives thereof or Sulfobutyl ether beta-cyclodextrin (SBE-beta-CD) or branched cyclodextrins or cyclodextrin polymers or suitable mixture thereof. In a preferred embodiment the suitable cyclodextrin variant is hydroxypropylated cyclo beta-dextrin (HP-β-CD).

In a preferred embodiment, the cyclodextrin or derivative is present in amount about 0.2% to about 10%. In another such embodiment, the amino acids which can be used as stabilizers or antioxidants include but are not limited to arginine, glycine, lysine, histidine, glutamic acid, aspartic acid, isoleucine, leucine, alanine, phenylalanine, tyrosine, tryptophan, methionine, serine, proline, cysteine/cystine and the like or suitable combination of any of the above. In a preferred embodiment, the suitable amino acid is methionine or cysteine or glycine or tryptophan or combination, thereof.

In an embodiment, the amino acid is present in amount about 0.01% to 10%.

Here, a skilled person can also use ascorbic acid or EDTA or combination, thereof, as an antioxidant(s) separately or in combination with other antioxidants) in the said formulation. The antioxidant according to the current invention is present in the concentration range of 0.001% to 1%, preferably, 0.01% to 0.5%.

In one of the embodiments, the stable liquid formulation comprises preservatives selected from hydroxybenzens (phenol, m-cresol, p-cresol, o-cresol, chlorocresol, benzyl alcohol and the like), paraben (methyl, ethyl, propyl, butyl and the like), sodium benzoate, benzyl benzoate, benzalkonium chloride, benzethonium chloride, sodium dehydroacetate and thimerosal, or mixtures thereof. In a preferred embodiment, the preservative is selected from phenol, paraben, sodium benzoate, benzyl benzoate and mixture thereof.

In a preferred embodiment, the preservative is present in amount about 0.01% to 1%. In a more preferred embodiment, the preservative is present in amount of 0.01% to 0.5%.

In another embodiment, the liquid formulation optionally comprises tonicity agents such as sodium chloride or potassium chloride. In a preferred embodiment, the tonicity agent is sodium chloride, which is present in amount about 10 mM to about 150 mM.

Phosphoric acid or sodium hydroxide can be used in a suitable amount to adjust the desired pH of the formulation.

The formulation may additionally further comprises one or more suitable other excipients, which are well known to a person skilled in the art.

In some embodiments, the liquid formulation maintains the storage stability in terms of not allowing any further protein degradation or modifications as compared to the initial.

In some embodiments, the liquid formulation maintains the stability during the process of formulation.

In one of the embodiments, the stable liquid formulation with said excipients can be prepared for combination of FSH and LH or FSH and hCG or LH and hCG.

To estimate the level of high molecular weight species or aggregates and low molecular weight or dissociated species, analytical HP-size exclusion chromatography was performed. To analyze oxidized species variants or purity of desired protein a person skilled in the art can use reversed-phase HPLC. In-vivo or in-vitro biological assay can be performed to check the biological activity of the desired protein. A person skilled in the art can use other analytical tools/techniques known in the art to check the physico-chemical as well as biological properties of the desired protein.

The said analytical methods used in the present invention are well known to a skilled person and a brief description of the same is provided below merely for the sake of reference only.

HP-Size Exclusion Chromatography (HP-SEC):

Samples were analyzed to estimate the high molecular weight species or aggregates and low molecular weight or dissociated species by HP-size exclusion chromatography (HP-SEC) using TSK gel G3000 SWXL column (7.8 mm I.D×30 cm L). Samples were loaded and eluted isocratically using sodium phosphate buffer at a flow rate of 0.5 mL/min. Elution was monitored at UV 215 nm.

The following non-limiting examples describe the different formulations, which can be prepared as per the present invention. It will be appreciated that other excipients may be added to these formulations and these are within the scope of a person skilled in the art.

The present invention is illustrated further through the following examples which are provided for illustration purpose and should not be construed as being a limitation to the scope of the invention.

### EXAMPLE 1

Active Ingredient

| | | |
|---|---|---|
| FSH | 44 | μg/mL |

Inactive Ingredients

| | | |
|---|---|---|
| Sodium phosphate | 10 | mM |

| | |
|---|---|
| Polyethylene Glycol | 0.1% |
| EDTA | 0.1% |
| Sodium benzoate | 0.3% |

Follitropin alfa was purified as per the technique known in the art. In this example, the purified Follitropin alfa was formulated in sodium phosphate buffer, further comprising polyethylene glycol, EDTA and sodium benzoate at desired concentrations, as described above. pH of the formulation medium was maintained around pH 7.0. If required, pH of the formulation can be adjusted using O-phosphoric acid or sodium hydroxide. Excipients were added to the protein solution from respective stock solutions to adjust the final concentration and volume was made up to the desired level. The formulated bulk was distributed in suitable container-closure systems (like vials, cartridges, syringes etc.) for storage. Similarly, a person skilled in the art can also formulate the composition for Follitropin beta. Upon formulation, samples were analyzed for the presence of high molecular weight species or aggregates and dissociated or fragmented species variants by HP-Size exclusion chromatography (HP-SEC). A person skilled in the art can analyze said parameters at various temperature conditions like Real-Time storage condition (between +2° C. and +8° C.), Accelerated storage condition (about +25° C.) or stressed condition (higher temperature).

### EXAMPLE 2

Active Ingredient

| | | |
|---|---|---|
| FSH | 44 | μg/mL |

Inactive Ingredients

| | | |
|---|---|---|
| Sodium phosphate | 10 | mM |

| | |
|---|---|
| Arginine | 0.5% |
| Polyethylene Glycol | 0.1% |
| EDTA | 0.1% |
| Sodium benzoate | 0.3% |

Follitropin alfa was purified as per the technique known in the art. In this example, the purified Follitropin alfa was formulated in sodium phosphate buffer, further comprising arginine, polyethylene glycol, EDTA and sodium benzoate at desired concentrations, as described above. pH of the formulation medium was maintained around pH 7.0. If required, pH of the formulation can be adjusted using O-phosphoric acid or sodium hydroxide. Excipients were added to the protein solution from respective stock solutions to adjust the final concentration and volume was made up to the desired level. The formulated bulk was distributed in suitable container-closure systems (like vials, cartridges, syringes etc.) for storage. Similarly, a person skilled in the art can also formulate the composition for Follitropin beta. Upon formulation, samples were analyzed for the presence of high molecular weight species or aggregates and dissociated or fragmented species variants by HP-Size exclusion chromatography. A person skilled in the art can analyze said parameters at various temperature conditions like Real-Time storage condition (between +2° C. and +8° C.), Accelerated storage condition (about +25° C.) or stressed condition (higher temperature).

### EXAMPLE 3

Active Ingredient

| | | |
|---|---|---|
| FSH | 44 | μg/mL |

Inactive Ingredients

| | | |
|---|---|---|
| Sodium phosphate | 10 | mM |

| | |
|---|---|
| Methionine | 0.01% |
| Polyethylene Glycol | 0.1% |
| EDTA | 0.1% |
| Sodium benzoate | 0.3% |

Follitropin alfa was purified as per the technique known in the art. In this example, the purified Follitropin alfa was formulated in sodium phosphate buffer, further comprising methionine, polyethylene glycol, EDTA and sodium benzoate at desired concentrations, as described above. pH of the formulation medium was maintained around pH 7.0. If required, pH of the formulation can be adjusted using O-phosphoric acid or sodium hydroxide. Excipients were added to the protein solution from respective stock solutions to adjust the final concentration and volume was made up to the desired level. The formulated bulk was distributed in suitable container-closure systems (like vials, cartridges, syringes etc.) for storage. Similarly, a person skilled in the art can also formulate the composition for Follitropin beta. Upon formulation, samples were analyzed for the presence of high molecular weight species or aggregates and dissociated or fragmented species variants by HP-Size exclusion chromatography. A person skilled in the art can analyze said parameters at various temperature conditions like Real-Time storage condition (between +2° C. and +8° C.), Accelerated storage condition (about +25° C.) or stressed condition (higher temperature).

### EXAMPLE 4

Active Ingredient

| | | |
|---|---|---|
| FSH | 44 | μg/mL |

Inactive Ingredients

| | | |
|---|---|---|
| Sodium phosphate | 10 | mM |

| | |
|---|---|
| Sucrose | 6% |
| Methionine | 0.01% |
| Polyethylene Glycol | 0.1% |
| EDTA | 0.1% |
| Phenol | 0.3% |

Follitropin alfa was purified as per the technique known in the art. In this example, the purified Follitropin alfa was formulated in sodium phosphate buffer, further comprising sucrose, methionine, polyethylene glycol, EDTA and phenol at desired concentrations, as described above. pH of the formulation medium was maintained around pH 7.0. If required, pH of the formulation can be adjusted using O-phosphoric acid or sodium hydroxide. Excipients were added to the protein solution from respective stock solutions to adjust the final concentration and volume was made up to the desired level. The formulated bulk was distributed in suitable container-closure systems (like vials, cartridges, syringes etc.) for storage. Similarly, a person skilled in the art can also formulate the composition for Follitropin beta. Upon formulation, samples were analyzed for the presence of high molecular weight species or aggregates and dissociated or fragmented species variants by HP-Size exclusion chromatography. A person skilled in the art can analyze said parameters at various temperature conditions like Real-Time storage condition (between +2° C. and +8° C.), Accelerated storage condition (about +25° C.) or stressed condition (higher temperature).

### EXAMPLE 5

Active Ingredient

| | | |
|---|---|---|
| FSH | 44 | μg/mL |

Inactive Ingredients

| Sodium phosphate | 10 | mM |
|---|---|---|
| Polyethylene Glycol | 0.1% | |
| Sodium chloride | 100 | mM |

| | |
|---|---|
| EDTA | 0.1% |
| Sodium benzoate | 0.3% |

Follitropin alfa was purified as per the technique known in the art. In this example, the purified Follitropin alfa was formulated in sodium phosphate buffer, further comprising polyethylene glycol, sodium chloride, EDTA and sodium benzoate at desired concentrations, as described above. pH of the formulation medium was maintained around pH 7.0. If required, pH of the formulation can be adjusted using O-phosphoric acid or sodium hydroxide. Excipients were added to the protein solution from respective stock solutions to adjust the final concentration and volume was made up to the desired level. The formulated bulk was distributed in suitable container-closure systems (like vials, cartridges, syringes etc.) for storage. Similarly, a person skilled in the art can also formulate the composition for Follitropin beta. Upon formulation, samples were analyzed for the presence of high molecular weight species or aggregates and dissociated or fragmented species variants by HP-Size exclusion chromatography. A person skilled in the art can analyze said parameters at various temperature conditions like Real-Time storage condition (between +2° C. and +8° C.), Accelerated storage condition (about +25° C.) or stressed condition (higher temperature).

Follitropin alfa is formulated in different compositions as described above and exposed to higher temperature to check degradation over the period of time. Results of HP-SEC analysis, obtained for the different samples are shown below in Table 1. No significant increase in either high molecular weight species (HMWs) or aggregates and low molecular weight (LMWs) or dissociated species observed over the period of time when Follitropin alfa is exposed to higher temperature with formulation compositions as described with different examples above.

TABLE 1
Results of HP-SEC analysis obtained with samples
of different formulation compositions

| | Exposed to higher temperature (40° C.) | | | | |
|---|---|---|---|---|---|
| Formulations | | Initial | 3^{rd} Day | 7^{th} Day | 15^{th} Day |
| Example 1 | HMWs/Aggregates | 0.03% | 0.02% | 0.02% | — |
| | Principal peak (/β intact) | 99.97% | 99.98% | 99.98% | — |
| | Dissociated species/LMWs | ND | ND | ND | — |
| Example 2 | HMWs/Aggregates | 0.01% | 0.03% | 0.02% | 0.02% |
| | Principal peak (/β intact) | 99.99% | 99.97% | 99.98% | 99.94% |
| | Dissociated species/LMWs | ND | ND | ND | 0.04% |
| Example 3 | HMWs/Aggregates | 0.03% | 0.02% | 0.03% | 0.02% |
| | Principal peak (/β intact) | 99.97% | 99.98% | 99.97% | 99.91% |
| | Dissociated species/LMWs | ND | ND | ND | 0.07% |
| Example 4 | HMWs/Aggregates | 0.05% | 0.06% | 0.05% | 0.02% |
| | Principal peak (/β intact) | 99.95% | 99.94% | 99.83% | 98.81% |
| | Dissociated species/LMWs | ND | ND | 0.12% | 1.18% |
| Example 5 | HMWs/Aggregates | 0.05% | 0.02% | 0.02% | — |
| | Principal peak (/β intact) | 99.95% | 99.98% | 99.98% | — |
| | Dissociated species/LMWs | ND | ND | ND | — |

ND—Not detectable;
HMWs—High molecular weight species;
LMWs—Low molecular weight species

Various compositions as mentioned in the preceding description and examples can also be prepared for FSH or its variant protein using similar process. FSH protein formulated with different compositions described in the present invention can also be stored between +2° C. and +8° C. for long term storage in suitable container-closure systems (like vials, cartridges, syringes etc.).

TABLE 2
Formulation compositions of Follitropin

| Example 6 | | | Example 7 | | |
|---|---|---|---|---|---|
| Active Ingredient | | | Active Ingredient | | |
| Follitropin | 44 | μg/mL | Follitropin | 44 | μg/mL |
| Inactive Ingredients | | | Inactive Ingredients | | |
| Sodium phosphate | 10 | mM | Sodium phosphate | 10 | mM |

| | | | |
|---|---|---|---|
| Sucrose | 6% | Methionine | 0.01% |
| Polyethylene Glycol | 0.1% | Sucrose | 6% |
| EDTA | 0.1% | Polyethylene Glycol | 0.1% |
| Phenol | 0.3% | EDTA | 0.1% |
| | | Phenol | 0.3% |

Results of HP-SEC analysis, obtained for different samples-stored between +2° C. and +8° C. are shown below in Table 3. No significant increase in either high molecular weight species (HMWs) or aggregates and low molecular weight (LMWs) or dissociated species observed over the period of time when Follitropin is stored at +2° C. to +8° C. with different formulation composition exemplified in Table 2.

TABLE 3
Results of HP-SEC analysis obtained with samples
of different formulation compositions

| | Stored between +2° C. and +8° C. | | |
|---|---|---|---|
| Formulations | | Initial | After 12 Months |
| Example 6 | HMWs/Aggregates | 0.2% | 0.2% |
| | Principal peak (/β intact) | 99.8% | 99.7% |
| | Dissociated species/LMWs | ND | 0.01% |
| Example 7 | HMWs/Aggregates | 0.4% | 0.3% |
| | Principal peak (/β intact) | 99.5% | 99.7% |
| | Dissociated species/LMWs | ND | 0.01% |

ND—Not detectable;
HMWs—High molecular weight species;
LMWs—Low molecular weight species

Similarly, a person skilled in the art can also formulate the other gonadotropins such as LH or HCG according to the present invention.

Various compositions can be prepared using the excipients disclosed in the specification and following similar processes as mentioned in the preceding Examples. The other suitable compositions that can be used for stabilization of the FSH or its variant protein are mentioned in the below Table 3.

TABLE 3

| Example 8 | Example 9 | Example 10 |
|---|---|---|
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.01-5% Polyethylene Glycol | 0.01-5% Polyethylene Glycol | 0.01-5% Polyethylene Glycol |
| 0.01-5% EDTA | 0.01-5% EDTA | 0.01-5% EDTA |
| 0.1%-0.5% Phenol | 0.1%-0.5% Phenol | 0.1%-0.5% Phenol |
| Example 11 | Example 12 | Example 13 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.01-5% Polyethylene Glycol | 0.01-5% Polyethylene Glycol | 0.1-10% Tryptophan |
| 0.01-5% EDTA | 0.01-5% EDTA | 0.01-5% Polyethylene Glycol |
| 0.1%-0.5% Phenol | 0.1%-0.5% Phenol | 0.01-5% EDTA |
| | | 0.1%-0.5% Phenol |
| Example 14 | Example 15 | Example 16 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 5-100 mM Methionine | 5-100 mM Cysteine | 5-100 mM Tryptophan |
| 0.001-1% Polysorbate 20 | 0.001-1% Polysorbate 20 | 0.001-1% Polysorbate 20 |
| 0.01-5% EDTA | 0.01-5% EDTA | 0.01-5% EDTA |
| 0.1%-0.5% Phenol | 0.1%-0.5% Phenol | 0.1%-0.5% Phenol |
| Example 17 | Example 18 | Example 19 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.001-1% Polysorbate 20 | 0.001-1% Polysorbate 20 | 0.1-10% Tryptophan |
| 0.01-5% EDTA | 0.01-5% EDTA | 00.001-1% Polysorbate 20 |
| 0.1%-0.5% Phenol | 0.1%-0.5% Phenol | 0.01-5% EDTA |
| | | 0.1%-0.5% Phenol |
| Example 20 | Example 21 | Example 22 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.01-5% Polyethylene Glycol | 0.01-5% Polyethylene Glycol | 0.01-5% Polyethylene Glycol |
| 0.01-5% EDTA | 0.01-5% EDTA | 0.01-5% EDTA |
| 0.1%-0.5% Paraben | 0.1%-0.5% Paraben | 0.1%-0.5% Paraben |
| Example 23 | Example 24 | Example 25 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.01-5% Polyethylene Glycol | 0.01-5% Polyethylene Glycol | 0.1-10% Tryptophan |
| 0.01-5% EDTA | 0.01-5% EDTA | 0.01-5% Polyethylene Glycol |
| 0.1%-0.5% Paraben | 0.1%-0.5% Paraben | 0.01-5% EDTA |
| | | 0.1%-0.5% Paraben |
| Example 26 | Example 27 | Example 28 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.001-1% Polysorbate 20 | 0.001-1% Polysorbate 20 | 0.001-1% Polysorbate 20 |
| 0.01-5% EDTA | 0.01-5% EDTA | 0.01-5% EDTA |
| 0.1%-0.5% Paraben | 0.1%-0.5% Paraben | 0.1%-0.5% Paraben |
| Example 29 | Example 30 | Example 31 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.001-1% Polysorbate 20 | 0.001-1% Polysorbate 20 | 0.1-10% Tryptophan |
| 0.01- 5% EDTA | 0.01-5% EDTA | 00.001-1% Polysorbate 20 |
| 0.1%-0.5% Paraben | 0.1%-0.5% Paraben | 0.01-5% EDTA |
| | | 0.1%-0.5% Paraben |
| Example 32 | Example 33 | Example 34 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.01-5% Polyethylene Glycol | 0.01-5% Polyethylene Glycol | 0.01-5% Polyethylene Glycol |
| 0.01-5% EDTA | 0.01-5% EDTA | 0.01-5% EDTA |
| 0.1%-0.5% Sodium | 0.1%-0.5% Sodium | 0.1%-0.5% Sodium |
| Benzoate | Benzoate | Benzoate |
| Example 35 | Example 36 | Example 37 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.01-5% Polyethylene Glycol | 0.01-5% Polyethylene Glycol | 0.1-10% Tryptophan |
| 0.01-5% EDTA | 0.01-5% EDTA | 0.01-5% Polyethylene Glycol |
| 0.1%-0.5% Sodium | 0.1%-0.5% Sodium | 0.01-5% EDTA |
| Benzoate | Benzoate | 0.1%-0.5% Sodium |
| | | Benzoate |
| Example 38 | Example 39 | Example 40 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.001-1% Polysorbate 20 | 0.001-1% Polysorbate 20 | 0.001-1% Polysorbate 20 |
| 0.01-5% EDTA | 0.01-5% EDTA | 0.01-5% EDTA |
| 0.1%-0.5% Sodium | 0.1%-0.5% Sodium | 0.1%-0.5% Sodium |
| Benzoate | Benzoate | Benzoate |
| Example 41 | Example 42 | Example 43 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.001-1% Polysorbate 20 | 0.001-1% Polysorbate 20 | 0.1-10% Tryptophan |
| 0.01-5% EDTA | 0.01-5% EDTA | 00.001-1% Polysorbate 20 |
| 0.1%-0.5% Sodium | 0.1%-0.5% Sodium | 0.01-5% EDTA |
| Benzoate | Benzoate | 0.1%-0.5% Sodium |
| | | Benzoate |
| Example 44 | Example 45 | Example 46 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.01-5% Polyethylene Glycol | 0.01-5% Polyethylene Glycol | 0.01-5% Polyethylene Glycol |
| 0.01-5% EDTA | 0.01-5% EDTA | 0.01-5% EDTA |
| 0.1%-0.5% Benzyl Benzoate | 0.1%-0.5% Benzyl Benzoate | 0.1%-0.5% Benzyl Benzoate |
| Example 47 | Example 48 | Example 49 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.01-5% Polyethylene Glycol | 0.01-5% Polyethylene Glycol | 0.1-10% Tryptophan |
| 0.01-5% EDTA | 0.01-5% EDTA | 0.01-5% Polyethylene Glycol |
| 0.1%-0.5% Benzyl Benzoate | 0.1%-0.5% Benzyl Benzoate | 0.01-5% EDTA |
| | | 0.1%-0.5% Benzyl Benzoate |
| Example 50 | Example 51 | Example 52 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Cysteine | 0.1-10% Tryptophan |
| 0.001-1% Polysorbate 20 | 0.001-1% Polysorbate 20 | 0.001-1% Polysorbate 20 |
| 0.01-5% EDTA | 0.01-5% EDTA | 0.01-5% EDTA |
| 0.1%-0.5% Benzyl Benzoate | 0.1%-0.5% Benzyl Benzoate | 0.1%-0.5% Benzyl Benzoate |
| Example 53 | Example 54 | Example 55 |
| 5-200 μg/mL FSH | 5-200 μg/mL FSH | 5-200 μg/mL FSH |
| 5-100 mM Sodium | 5-100 mM Sodium | 5-100 mM Sodium |
| phosphate buffer of pH 7.0 | phosphate buffer of pH 7.0 | phosphate buffer of pH 7.0 |
| 0.1-10% Sucrose | 0.1-10% Sucrose | 0.1-10% Sucrose |
| 0.1-10% Methionine | 0.1-10% Methionine | 0.1-10% Methionine |
| 0.1-10% Cysteine | 0.1-10% Tryptophan | 0.1-10% Cysteine |
| 0.001-1% Polysorbate 20 | 0.001-1% Polysorbate20 | 0.1-10% Tryptophan |
| 0.01-5% EDTA | 0.01-5% EDTA | 00.001-1% Polysorbate 20 |
| 0.1%-0.5% Benzyl Benzoate | 0.1%-0.5% Benzyl Benzoate | 0.01-5% EDTA |
| | | 0.1%-0.5% Benzyl Benzoate |

Similar formulations can be prepared using 5-100 mM of acetate buffer (sodium acetate-acetic acid) or of succinate buffer or of citrate buffer (sodium citrate-citric acid) or of Phosphate buffered saline or of Arginine buffer or of citrate-phosphate buffer or of histidine buffer and the like with pH range of about pH 5.0 to pH 9.0.

Similar formulation can be prepared using 0.01% to 10% of raffinose or of trehalose or of sorbitol or of dextran or of cyclodextrin or of mannitol.

Similar formulation can be prepared using 0.001% to 5% of pluronics (poloxamers) alone or in combination with Polyethylene Glycol or polysorbates.

Similar formulation can be prepared using ascorbic acid in a suitable concentration.

Similar formulation can be prepared using tonicity agents with suitable concentrations.

Similar formulation can also be prepared for other gonadotropins, like LH or its variants and hCG or its variants or combination thereof. A skilled person can prepare similar formulation for combination of gonadotropins selected from LH or its variant and FSH or its variant, hCG or its variant and FSH or its variant.

The formulations of the present invention can be used for the treatment in which activity of gonadotropin is detrimental.

1-28. (canceled)

29. A liquid pharmaceutical formulation comprising an effective amount of gonadotropin in a buffer system, polyethylene glycol as a stabilizer, suitable preservative and optionally other suitable excipients.

30. The formulation as claimed in claim 29, wherein the buffer system is selected from histidine-buffer, arginine-buffer, citrate-buffer, succinate-buffer acetate-buffer, phosphate-buffer, tromethamine buffers, or citrate-phosphate buffer or a mixture thereof, wherein the buffer is in a concentration of 5 mM to about 100 mM.

31. The formulation as claimed in claim 29, wherein the polyethylene glycol is present in the concentration of 0.005% to 10%.

32. The formulation as claimed in claim 29, wherein the preservative is selected from hydroxybenzenes (phenol, m-cresol, p-cresol, o-cresol, chlorocresol, benzyl alcohol and the like), paraben (methyl, ethyl, propyl, butyl and the like), sodium benzoate, benzyl benzoate, benzalkonium chloride, benzethonium chloride, sodium dehydroacetate and thimerosal or mixtures thereof preferably, sodium benzoate or phenol wherein the preservative is present in a concentration of 0.01% to 1%.

33. The formulation as claimed in claim 29, wherein other suitable excipients can be selected from antioxidants, other stabilizers, surfactants, tonicity agents and suitable combination thereof.

34. The formulation as claimed in claim 33, wherein the antioxidant is selected from EDTA, ascorbic acid or a combination thereof, wherein the antioxidant is present in the concentration of 0.001% to 1.

35. The formulation ax claimed in claim 33, wherein the surfactant is selected from polyoxyethylensorbitan fatty acid esters (Tween), polyoxyethylene alkyl ethers, alkylphenylpolyoxyethylene ethers, polyoxyethylene-polyoxypropylene copolymer and sodium dodecyl sulphate (SDS), preferably polysorbate 80 or polyoxyethylene-polyoxypropylene copolymer, wherein the surfactant is present in the concentration of 0.001% to about 1%.

36. The formulation as claimed in claim 33, wherein the tonicity agent is sodium chloride or potassium chloride wherein the tonicity agent is present in amount about 10 mM to about 150 mM.

37. The formulation as claimed in claim 33, wherein the stabilizer is selected from suitable sugars, polyols, amino acids or a combination thereof.

38. The formulation as claimed in claim 37, wherein the amino acid is selected from arginine, glycine, lysine, histidine, glutamic acid, aspartic acid, isoleucine, leucine, alanine, phenylalanine, tyrosine, tryptophan, methionine, serine, proline, cysteine/cystine and suitable combination thereof wherein the amino acid is present in the concentration of 0.01% to 1%.

39. The formulation as claimed in claim 37, wherein the sugar is selected from glucose, fructose, galactose, mannose, sorbose, ribose, deoxyribose, dextran, dextrin, sucrose, trehalose, lactose, maltose, raffinose and suitable mixtures thereof, wherein the sugar is present in the concentration of 0.005% to 10%.

40. The formulation as claimed in claim 29, wherein the concentration of gonadotropin is between 5 μg/ml to 200 μg/ml or wherein the pH of the formulation is between pH 6 and pH 8.

41. A formulation as claimed in claim 29, comprising:
a) 5-200 μg/ml of gonadotropin;
b) 0.005-10% polyethylene glycol;
c) a buffer system for maintaining the pH between 6 and 8;
d) 0.01-1% of preservative; and
e) optionally with other suitable excipients.

42. The formulation as claimed in claim 41, wherein the buffer system is a phosphate of succinate or acetate or citrate-phosphate buffer system and the preservative is sodium benzoate or phenol or m-cresol.

43. The formulation as claimed in claim 41, which further comprises a surfactant and/or tonicity agent selected from polysorbate 80 and/or sodium chloride respectively.

44. The formulation as claimed in claim 29, wherein the gonadotropin is selected from follicle stimulating hormone or variant thereof luteinizing hormone or variant thereof, human chorionic gonadotropin hormone or variant thereof and combination thereof.

45. The formulation as claimed in claim 44, wherein the follicle stimulating hormone is follitropin alfa or Follitropin beta or both.

46. The formulation as claimed in claim 41, further comprises antioxidant selected from EDTA, ascorbic acid and combination thereof.

47. A formulation as claimed in claim 29, comprising:
a) 44 μg/ml of follitropin alfa;
b) 0.1% polyethylene glycol;
c) 10 mM phosphate butler with a pH about 7;
d) 0.1% of EDTA;
e) 0.3% of sodium benzoate; and
f) 100 mM of sodium chloride.
